(19) United States
(12) Patent Application Publication
(10) Pub. No.: US 2008/0095882 A1
(43) Pub. Date: Apr. 24, 2008

(54) Controlled Spoilage Food Compositions

(75) Inventors: Michael E. Stiles, Edmonton (CA); Lynn McMullen, Edmonton (CA); David C. Smith, Toronto (CA)

Correspondence Address: WILLIAM J BUNDREN;THE LAW OFFICE OF WILLIAM J BUNDREN, 734 LaRue Road, Millersville, MD, 21108, US

(73) Assignee: CANBIOCIN, INC., Alberta (CA)

(21) Appl. No.: 10/519,231

(22) PCT Filed: Jun. 27, 2003
(86) PCT No.: PCT/CA03/00986

§ 371 Date: Mar. 14, 2006

Related U.S. Application Data
(60) Provisional application No. 60/391,645, filed on Jun. 27, 2002.

(51) Int. Cl. A23L 3/3571; A23L 1/314; C12N 1/20

(52) U.S. Cl. 426/9; 426/231; 426/56; 426/61; 435/252.1

(57) ABSTRACT

The present invention provides a method of controlling the development of resident spoilage and pathogenic bacteria in food products by introducing, into the food products, known bacteria that produce novel bacteriocins or metabolites which inhibit or kill the spoilage and pathogenic bacteria. Specifically, the method of the present invention comprises introducing, into meat products, known bacteria that produce novel bacteriocins or metabolites which inhibit or kill L. monocytogenes. Modifications are possible within the scope of the invention.

### FIELD OF INVENTION

The present invention relates to food compositions and to controlled spoilage in such compositions.

### BACKGROUND TO THE INVENTION

Between six and 30 million Americans become ill each year from microorganisms in their food, of which an estimated 9,000 die. It has been calculated that the costs of foodborne illness in North America represents between $4 and $14 billion annually in terms of medical expenses, lost wages, insurance costs and liability.

The presence of pathogenic bacteria in food products is a major concern to the food processing industry. In recent years, due to government regulations in USA, there is a zero tolerance of Listeria monocytogenes (“L. monocytogenes”) in ready-to-eat meats. The presence of L. monocytogenes in food has lead to numerous product recalls and in some instances temporary plant closures.

Consumers expect foods to be available year round, free of pathogens, and have a long storage life. Consumer trends encourage “natural” products that are free from artificial preservatives and are minimally processed. The inherent properties of foods and how they are packaged (heat treatment, water activity, pH, storage temperature, redox potential, packaging atmosphere and composition) have been employed to build safety “hurdles” in minimally processed foods to extend storage life, and, more importantly, to block growth of foodborne pathogenic bacteria. A technology that would result in the predictable spoilage of the food with the ability to further inhibit the growth of pathogenic bacteria without adversely affecting consumer acceptability of the product would greatly enhance the safety of the foods during prolonged refrigerated storage.

While uncontrolled bacterial growth can cause great material damage to the food, there is a potential danger to consumer health if potentially pathogenic bacteria are present and uncontrolled microbial growth occurs in food. In specific cases, this may have grave consequences for the manufacturer of the products, from loss of company image and product recalls to claims for compensation. The potential market of many minimally processed food products is thus severely restricted by uncontrolled microbial spoilage. As the use of minimal food-processing technologies widens, there is a need to develop natural consumer-friendly methods to prevent product deterioration and growth of bacterial pathogens to achieve a predictable storage life and product safety.

The patent literature contains several proposals with respect to solving this problem. Cells of lactic acid bacteria have been combined with the food substance to provide a food mixture containing about 10⁵ to 10⁸ Colony Forming Units per gram (“CFU/g”) the food mixture, or about 0.1 to 1.0 percent weight (“wt %”) lactic acid bacteria cells based on the total weight of the food mixture. The cell count of the lactic acid bacteria fraction, at the time it is combined with the food substance, preferably does not increase by more than about 10 to 100%, more preferably 10 to 50%, as part of the food mixture. In this case, the bacteriocin-producing bacterium was a Pediococcus sp. (U.S. Pat. No. 5,186,962) and does not grow at refrigeration temperatures.

Another approach involved a novel bacteriocin (piscicolin 126) having a range of activity different to and preferably narrower than those of nisin and pediocin PA-1. The invention consisted of a substantially pure preparation of the bacteriocin having a molecular mass of about 4.4 kDa and a specific antimicrobial activity toward other bacteria. The patent also claimed that a specific amino acid sequence was responsible for the antimicrobial nature of the bacteriocin and that the initial bacteriocin was isolated from Carnobacterium piscicola strain JG126 (U.S. Pat. No. 6,054,163).

Another approach involved an antimicrobial composition comprising a Streptococcus-derived or Pediococcus-derived bacteriocin or synthetic equivalent antibacterial agents in combination with a chelating agent. Such composition was used in conjunction with a foodstuff or with a food packaging film (with or without chelating agent) to protect foodstuffs against the growth of harmful bacteria, such as Listeria. The patent also disclosed methods of protecting foodstuffs using film having a transferable antimicrobial agent which may protect food stuff surfaces before and/or following removal of the film and peelable films useful in such methods which include the above bacteriocins (U.S. Pat. No. 5,573,801).

Another approach involved a method of inhibiting L. monocytogenes in a food or other material which can be contaminated with this pathogen using a bacteriocin produced by DNA in Pediococcus acidilactici. The bacteriocin was produced in Pediococcus acidilactici containing a 6.2 Mdal (9.4 Kilobase pairs) plasmid encoding for the bacteriocin (U.S. Pat. No. 4,929,445).

Another approach involved a method of inhibiting foodborne pathogenic and spoilage microorganisms in processed foods using Lactobacillus sp., which produces an antimicrobial substance at refrigeration temperature. The method was particularly effective in inhibiting gas producing heterofermentative spoilage microorganisms, mold, foodborne pathogenic bacteria (Listeria and Salmonella) and psychrotrophic microorganisms that can occur in processed foods (U.S. Pat. No. 4,874,704).

Another approach involved a metabolite(s) of Propionibacterium sp., having a metabolite of molecular weight greater than 300 added to a food product to inhibit the growth of gram-negative psychrotrophic bacteria, yeasts, mold, gram-positive bacteria, including Listeria. The metabolite material may contain less than 0.02% propionic acid such that there was insufficient propionic acid per se to inhibit microbial growth. The metabolite was produced by growing Propionibacterium in a liquid growth medium to produce a mixture containing the metabolic material(s). The mixture can be concentrated and added to a food product as a concentrated liquid or powder. The metabolite material added to a food may contain viable cells of Propionibacterium sp. (U.S. Pat. No. 5,096,718).

Another approach involved an atypical Bacillus sublilis strain NRRL B-21974 from Pozol, a Mexican beverage used in controlling molds and other spoilage microflora in various materials, particularly foods including dough, tortillas, moist grains and cheese. The Bacillus subtilis can be used in living or non-living form in the materials. The materials can include packaging for foods (U.S. Pat. No. 5,919,695).

Another approach involved a novel strain, Lactobacillus sp. AS-1A (ATCC No. 69890), described for use to inhibit the growth of bacteria in foods, particularly at refrigeration temperatures. Lactobacillus sp. AS-1A (ATCC 69890) is particularly effective in inhibiting bacteria present in raw milk and pasteurized milk (U.S. Pat. No. 5,759,843).

Another approach involved the use of a novel antimicrobial agent. More particularly, a novel bacteriocin with nisin-like properties. The bacteriocin is designated lacticin 3147 and has the following properties: a molecular weight of approximately 2.8 kDa; inhibiting activity against Lactococci, Lactobacilli, Enterococci, Bacilli, Leuconostoc, Pediococci, Clostridia, Staphylococci and Streptococci; sensitivity to the proteases trypsin, alpha-chymotrypsin, proteinase K and pronase E but not pepsin; heat-stability; activity at acidic pH; and the capability of inhibiting nisin-producing bacterial strains (U.S. Pat. No. 6,207,411).

Another approach involved the use of a composition of matter which demonstrates efficacy against both gram-positive and gram-negative bacteria containing: (a) a gram-negative bacterium inhibiting effective amount of propionibacteria metabolites with the proviso that such metabolites not solely comprise propionic acid; (b) a gram-positive bacteria inhibiting effective amount of a lantibiotic; and (c) a chelating effective amount of one or more phosphate salts which function as a chelating agent to bind the propionibacteria metabolites and lantibiotics to the surface of the substrate being treated (U.S. Pat. No. 6,207,210).

Another approach involved a method for preserving a food product, such as a meat, comprising steps of inoculating meat with an effective amount of non-pathogenic, non-spoilage bacteria to competitively inhibit the growth of undesirable pathogenic and spoilage bacteria. Edible films that incorporate bacteria on the food product are used to ensure competitive inhibition of the spoilage and pathogenic bacteria (U.S. Pat. Nos. 6,039,984, and 5,869,113).

Another approach involved a method for preserving a food product, such as meat, comprising inoculating meat with an effective amount of non-pathogenic, non-spoilage bacteria to competitively inhibit the growth of undesirable pathogenic and spoilage bacteria. Preferably, either L. delbrueckii or Hafnia alvei bacteria are used to inoculate a meat product. Bacteria present on a meat product is first reduced to a number below about 5000 bacteria per gram of meat, e.g. by dehairing an animal and then spraying the meat with an organic acid prior to inoculation with bacteria. The meat product is then vacuum packaged and stored in a refrigerated environment of about 1° C. to about 7° C. Meat products preserved in accordance with the method of the invention can enjoy a refrigerated shelf life of up to about 150 days without surface discoloration or the generation of undesirable gaseous by-products (U.S. Pat. Nos. 5,576,035 and 5,374,433).

### SUMMARY OF INVENTION

In the present invention, live bacteria are added to a food product, such as fresh meat, to provide a controlled spoilage to the food product and to prevent the development of resident spoilage and pathogenic bacteria, such as L. monocytogenes. In essence, the resistant spoilage bacteria are replaced by a known spoilage bacterium to provide competitive inhibition, so that the spoilage of a food product is predictable and the shelf-life of a food product can be determined with accuracy knowing the amount of bacteria added.

### BRIEF DESCRIPTION OF DRAWINGS

FIGS. 1.1 to 1.3 show graphically the results of experiment with respect to protected spoilage by selected bacteria of regular BBQ frankfurters.

FIGS. 2.1 to 2.3 show graphically the antimicrobial activity of Carnobacterium piscicola cultures; and

FIG. 3, consisting of panels A to D, illustrates graphically the change in sensory characteristics of frankfurters inoculated with Carnobacterium piscicola and Leuconostoc gelidum.

### GENERAL DESCRIPTION OF INVENTION

An embodiment of the invention includes using a composition of the present invention to further protect a food product from the growth of gram-positive pathogenic bacteria, including but not limited to L. monocytogenes.

Another embodiment of the invention includes a method for treating a food product to give a predictable storage life. As used herein, predictable storage life refers to a known period in which the food product remains acceptable for human consumption. For example, predictable storage life includes applying Carnobacterium piscicola NCIMB 702852 or UAL26 to frankfurters to achieve a minimum storage life of 10 weeks at refrigeration temperatures.

An embodiment of the invention includes a method of treating fresh food by applying a microorganism, its pasteurized or unpasteurized fermentate, or combinations thereof to the food. In these embodiments of the invention the microorganism and its pasteurized or unpasteurized fermentate produce a predictable or controlled storage life.

Another embodiment of the invention is the use of selected natural bacteria to give a predictable storage life by treating the food with an amount of a natural bacterium that exceeds the level of natural contamination of the bacterium in the food.

In another embodiment of the invention, the composition applied to the food comprises one or more natural bacterial cultures, pasteurized or unpasteurized fermentate produced by the selected natural bacteria, or combinations thereof. In preferred embodiments of the invention, the food is treated with the combination of the natural bacteria and its pasteurized or unpasteurized fermentate. In the most preferred embodiment of the invention, the food is treated with the combination of selected natural bacteria and the bacterial pasteurized or unpasteurized fermentate of a selected natural bacterial culture.

In another embodiment of the invention, a method of predicting spoilage of a food product wherein a known spoilage bacterium is added to the food product to provide a bactericidal or bacteriostatic effect against L. monocytogenes.

As used herein, gram-positive pathogenic bacteria refer to, but are not limited to, Staphylococcus aureus, Enterococcus spp. and L. monocytogenes.

The procedures provided herein are applicable to all 13 known serotypes of L. monocytogenes. The compositions of the present invention are effective against strains of L. monocytogenes serotypes 1/2a, 1/2b, 3a and 4b. The spoilage bacteria used herein may be psychrotrophic. Specific spoilage bacteria that may be used include Carnobacterium piscicola NCIMB 702852 or UAL26, and Lactobacillus sakei UAL185. Combinations of two or more species may be used which are synergistic in action against the growth of L. monocytogenes.

The compositions of the present invention include one or more selected spoilage bacteria to achieve a predictable storage life and/or protect against the growth of pathogenic bacteria. The selected spoilage bacteria of the present invention include, but are not limited to, Carnobacterium piscicola NCIMB 702852, UAL26, CB1, CB2 and CB3, and Lactobacillus sakei UAL185. The method of the present invention includes the use of one or more natural bacterial cultures, homologous pasteurized or unpasteurized fermentate, heterologous pasteurized or unpasteurized fermentate, or combinations thereof. The natural bacterial cultures of the present invention are described above. A homologous fermentate refers to the culture supernatant of a single bacterial culture processed according to standard culture preparation techniques. A heterologous fermentate refers to the culture supernatant derived from a different bacterial culture processed according to standard culture preparation techniques. The homologous or heterologous fermentates may be pasteurized or unpasteurized, lyophilized or freeze dried. Two or more bacterial cultures may be mixed or added separately. Two or more bacterial fermentates may be mixed or added separately. A bacterial culture combined with one or more fermentates may be mixed, or added sequentially.

Combinations of two bacteria or pasteurized fermentate bacterial cultures may be used including combination of Carnobacterium piscicola NCIMB 702852 and/or Carnobacterium piscicola UAL26 and/or a heat resistant strain of Carnobacterium sp. and/or Carnobacterium divergens and/or Leuconostoc gelidum UAL187 and/or Lactobacillus sakei UAL185.

The compositions and methods of the present invention may also include additional additives or metabolites, which are bacteriocins or which are not bacteriocins, and not lactic acid or hydrogen peroxide.

Specific bacteriocins which may be used include bacteriocins of specific structure and molecular weight isolated from Carnobacterium piscicola NCIMB 702852 or UAL26, a heat resistant strain of Carnobacterium piscicola, Carnobacterium divergens, Leuconostoc gelidum UAL187 and Lactobacillus sakei UAL185. The bacteriocin may be produced by genetic engineering wherein the gene(s) encoding the bacteriocin is expressed from a microorganism. The spoilage bacteria used herein may be initially screened for high activity towards virulent L. monocytogenes. The spoilage bacteria are preferably selected such that the inhibitory activity of the bacteriocin is not affected by glutathione.

The packaged food product may be introduced with specific spoilage bacteria and stored under refrigeration conditions. During storage, the specific spoilage bacteria do not form carbon dioxide nor do they cause discoloration of meat. The procedure extends the “bloom” or red color of fresh meat products for a longer time than is observed with the resident spoilage bacteria. However, when color is used to determine storage life, the present invention extends the storage life of the product beyond that produced by the resident spoilage bacteria.

In another exemplary embodiment of the invention, the invention includes a method of preserving foods or beverages comprising the steps of adding to the food or beverage an effective amount of a bacterial culture of the present invention, alone or in combination with a fermentate, to the food or beverage. The inventors have found that an amount of 10² CFU/g or lower is not typically sufficient to compete with the existing adventitious microbial population. The inventors have found that greater than about 10³ CFU/g or higher are sufficient or overcome the growth of the existing adventitious microbial population. One skilled in the art will recognize that the amount of adventitious bacteria in a food product is variable; in accordance with the present invention, the amount of the composition should be about ten times or more higher than the amount of adventitious spoilage or pathogenic bacteria. The known spoilage bacteria bacterium usually is added to the food product in an amount greater than the natural levels of spoilage bacteria in the food product, generally about 10³ CFU to about 10⁴ CFU per gram of food product. As used herein, pre-determined storage refers to the capability of controlling spoilage for a discrete period, at which point spoilage becomes evident. For example, bacteria can be applied to a food product to attain a spoilage period of 10 weeks, at which point off-odors or off-flavors, such as sourness, may occur. Within the 10-week period, the composition of the present invention controls spoilage by one or more of the following: by applying bacteria having a known spoilage period; by applying bacteria that produces one or more bacteriocins that kill or control spoilage bacteria.

In accordance with the present invention, the composition has the added benefit of controlling or killing pathogenic bacteria, including but not limited to L. monocytogenes.

The known spoilage bacterium may be introduced to the food product in any convenient manner. In one procedure, the surface of the food product may be introduced with the known spoilage bacterium in the form of a live bacterial culture, a pasteurized or unpasteurized fermentate or with a combination of live bacterial culture and pasteurized or unpasteurized fermentate, which may be from the same or a different bacterium.

Alternatively, the protective live bacterial culture, protective pasteurized or unpasteurized fermentate of the bacterial culture, mixture of protective live bacterial culture and protective pasteurized or unpasteurized fermentate of the bacterial culture, bacteriocin or mixture with live bacterial culture, may be introduced to the food product by mixing with the food product.

Any other desired method of application of a liquid or powder to a substrate may be used, including dipping, spraying, mixing, injecting, tumbling and incorporation into a plastic film.

The food product to which the present invention is applied may vary widely and may be a cooked or uncooked food product. For example, the food product may be a cooked or cured ready-to-eat meat product, including tissues from poultry, beef, pork, lamb, goat and seafood.

The food product also may be a whole cooked vegetable or plant or a chopped or comminuted cooked vegetable or plant. The food product may be a fresh whole uncooked vegetable or plant or a chopped or comminuted cooked vegetable or plant. The food product also may be a non-pasteurized or pasteurized cheese, a batter, a grain or an egg or liquid egg.

The food product may be in packaged form. For meat products, modified-atmosphere packaging (MAP) with elevated levels of carbon dioxide, including vacuum packaging, has been employed. Such packaging may be used herein. The modified atmosphere may have gases in the range of nitrogen ≦70%, oxygen close to 0%, carbon dioxide ≧20%, preferably nitrogen 60%, oxygen 0% and carbon dioxide 40%. Another modified atmosphere that would be preferred would be 100% carbon dioxide. Alternatively, a modified atmosphere of 220% carbon dioxide and ≧50% oxygen would also be applicable for this application, preferably 20% carbon dioxide and 80% oxygen.

The known spoilage bacteria and related materials discussed above also may be used as a sanitizing agent and/or aerosol to provide an environment hostile to L. monocytogenes. For example, the known spoilage bacteria may be applied to brine chiller water used to cool hot dogs from the smoke house of a hot-dog factory. Another possible application would be to apply the spoilage bacteria and related materials discussed above to a drain to inhibit the growth of L. monocytogenes.

### EXAMPLES

### Example 1

This Example illustrates controlled spoilage by selected bacteria of regular BBQ frankfurters.

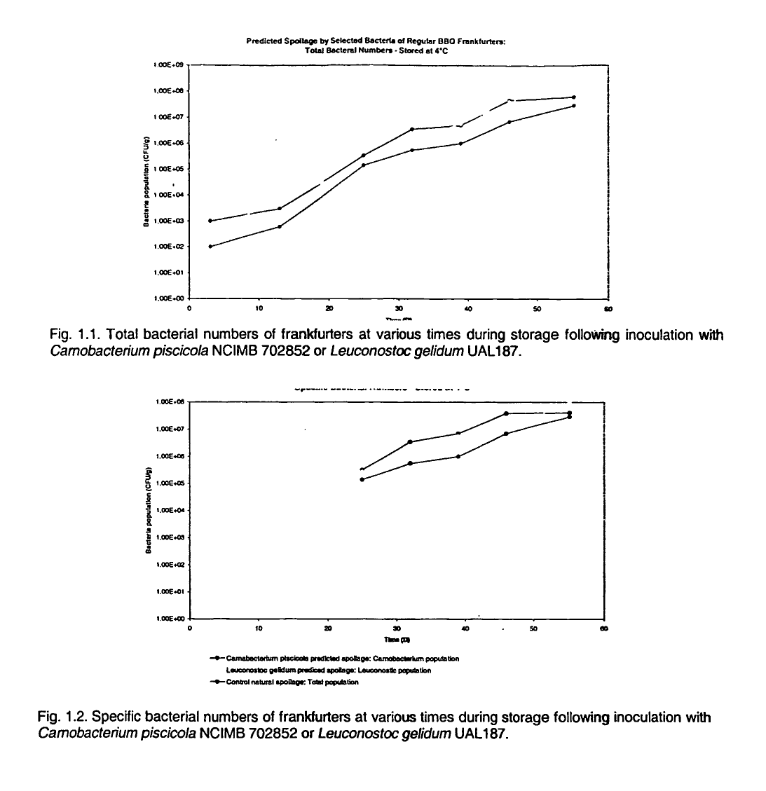

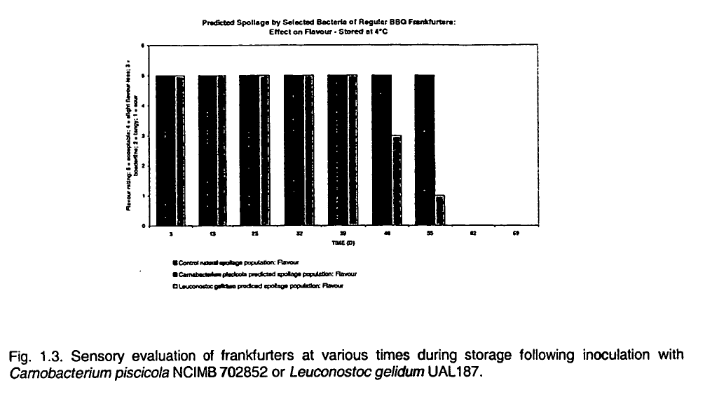

Freshly manufactured Regular BBQ frankfurters were inoculated by dipping in cultures of either Carnobacterium piscicola NCIMB 702852 or Leuconostoc gelidum UAL187. For the preparation of inocula, bacteria from frozen culture were subcultured twice in APT broth (Difco; Becton Dickinson) and incubated for 24 hours at 25° C. The cultures of Carnobacterium piscicola NCIMB 702852 or Leuconostoc gelidum UAL187 were standardized with sterile distilled water such that the dipped frankfurters were inoculated with preferably ≦10³ per cm². In the control samples, sterile water was substituted for the cultures. The inoculated frankfurters were dried on a sterile rack and vacuum packaged (2 per pack). The frankfurters were stored at 4° C. At the times indicated in FIGS. 1.1 to 1.3, packages of frankfurters were removed from storage and the total bacterial population, specific bacterial population and flavor were determined.

Samples were prepared for microbial analysis by excising a piece of frankfurter with flame sterilized scalpels. A ten-gram sample of frankfurter was placed in a sterile Stomacher bag, homogenized for 2 minutes with 90 ml of sterile 0.1% peptone water using a Colworth Stomacher 400 or similar. Bacterial numbers were enumerated by standard dilution and plating techniques. Total aerobic plate count was determined on Plate Count agar (PCA, Difco) incubated aerobically at 25° C. for 48 hours. Lactic acid bacteria counts were determined on Bacto APT agar (Difco) incubated anaerobically (BBL Anaerobic System with 5 to 10% CO₂) for 48 hours. Carnobacterium piscicola numbers were determined by difference on Lactobacilli MRS agar (Difco) for 48 hours. Acetate inhibits or retards the growth of Carnobacterium. Numbers of Leuconostoc gelidum were determined by difference on APT with sucrose added.

Bacterial numbers were enumerated by standard dilution and plating techniques. Total aerobic plate count was determined on Plate Count agar (PCA, Difco) incubated aerobically at 25° C. for 48 hours. Lactic acid bacteria counts were determined on Bacto APT agar (Difco) incubated anaerobically (BBL Anaerobic System with 5 to 10% CO₂) for 48 hours. Carnobacterium piscicola numbers were determined by difference on Lactobacilli MRS agar (Difco) for 48 hours. Acetate inhibits or retards the growth of Carnobacterium.

Bacterial numbers are reported as Colony Forming Units per gram (“CFU/g”).

Samples for sensory analysis were evaluated unheated at room temperature.

A trained panel was used to evaluate the sensory quality of the frankfurters throughout the storage period. The flavor of the inoculated frankfurters was compared with the control on a five point scale, 1=acceptable, 3=marginal, 5=unacceptable.

The results obtained are shown graphically in FIGS. 1.1 to 1.3. As may be seen therein, when the frankfurters were inoculated with Carnobacterium piscicola or Leuconostoc gelidum cells 10-fold higher than the natural bacterial flora then, predicted controlled spoilage by the inoculated bacterium ensued. However, only frankfurters inoculated with Carnobacterium piscicola showed predicted spoilage equivalent to the control without compromising the products flavor.

### Example 2

This Example illustrates antimicrobial activity of two strains of Carnobacterium piscicola.

Three compatible strains of L. monocytogenes (List4, HPB65 and HPB642) were grown separately, centrifuged and washed three times with sterile 0.85% saline and resuspended in sterile 0.85% saline for use as the “Listeria” inoculum “cocktail”. L. monocytogenes CDC 7662 was grown separately and inoculated as a single bacterial culture. The lactic acid bacteria (Carnobacterium piscicola UAL26 or UAL26/8A) were grown separately, centrifuged and washed three times with sterile 0.85% saline and resuspended separately in sterile 0.85% saline for use as the “lactic” inocula.

Freshly prepared, regular frankfurters were obtained from a meat processor in Edmonton and they were inoculated by immersion in the inoculum containing either the washed Listeria cocktail or the single L. monocytogenes culture, and the lactic acid bacterium. The inoculated frankfurters were dried on a sterile rack and vacuum packaged. The following treatments were prepared:

Un-inoculated control. Dipped in sterile 0.85% saline.

Inoculated control. Dipped in 0.85% saline containing the L. monocytogenes CDC 7662 or the L. monocytogenes cocktail to give ˜1000 CFU of Listeria per cm².

L. monocytogenes CDC 7662 or L. monocytogenes cocktail+Carnobacterium piscicola UAL26 to give 1000 CFU of Listeria and 10,000 CFU of Carnobacterium piscicola UAL26 per cm².

L. monocytogenes CDC 7662 or L. monocytogenes cocktail+Carnobacterium piscicola UAL26/8A to give 1000 CFU of Listeria and 10,000 CFU of Carnobacterium piscicola UAL26/8A per cm².

Two frankfurters from each treatment were aseptically transferred to a high barrier film plastic bag and vacuum packaged. The frankfurters were stored at 4° C. and sampled once per week (days 0, 7, 14, 21, 28, 35, 42, 49, 56, 63, 70).

Samples were prepared for microbial analysis by excising a piece of frankfurter with flame-sterilized scalpels to give a sample with 10 cm² surface area. The sample of the frankfurter was placed in a sterile Stomacher bag, homogenized for 2 minutes with 90 ml of sterile 0.1% peptone water using a Colworth Stomacher 400 or similar.

Bacterial numbers were enumerated by standard dilution and plating techniques. Total aerobic plate count was determined on Plate Count agar (PCA, Difco) incubated aerobically at 25° C. for 48 hours. Lactic acid bacterial numbers were determined on Bacto APT agar (Difco) incubated anaerobically (BBL Anaerobic System with 5 to 10% CO₂) at 25° C. for 48 hours. Listeria monocytogenes counts were determined on PALCAM Agar Base (Oxoid, Unipath Ltd., England) supplemented with selective supplement (SR150E, Oxoid). Plates were incubated at 37° C. for 24 h and enumerated.

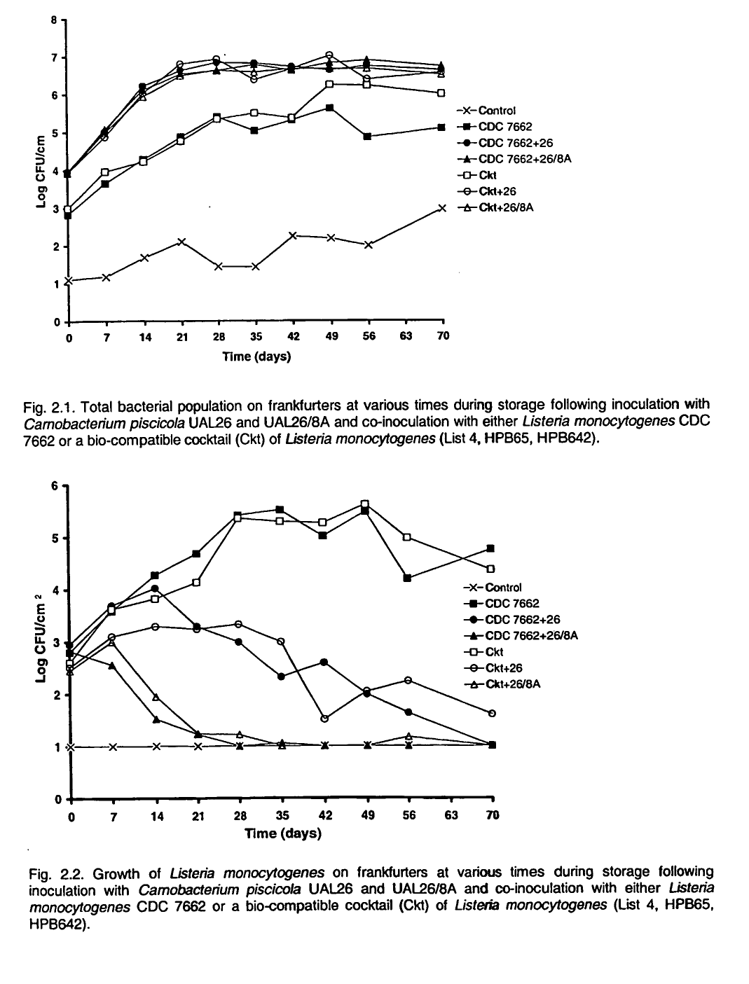

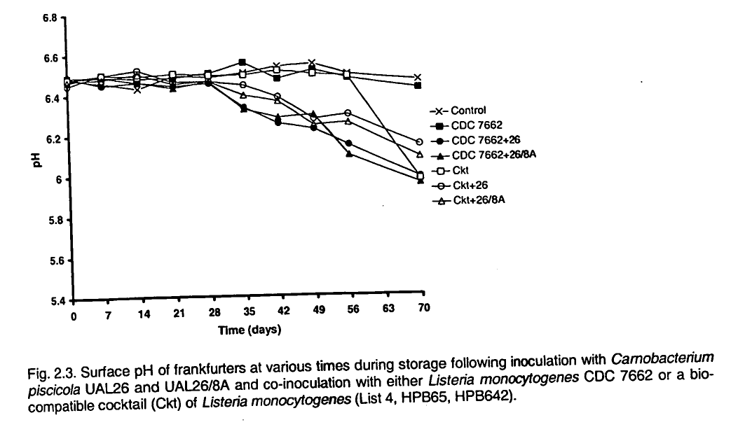

Bacterial numbers are reported as logarithms (“log10”) Colony Forming Units per gram (“LOG CFU/g”). The results obtained are shown graphically in FIGS. 2.1 to 2.3. As may be seen therein, when the frankfurters were inoculated with the Carnobacterium UAL26 or UAL26/8A at a level that was 10-fold higher than the Listeria cocktail or L. monocyotenes CDC 7662 the controlled predicted spoilage population inhibited the growth of L. monocytogenes.

### Example 3

This example illustrates the impact of the growth of Carnobacterium piscicola and Leuconostoc gelidum on the sensory characteristics of vacuum packaged frankfurters.

The following laboratory-scale study was designed to investigate the sensory characteristics of Carnobacterium piscicola and Leuconostoc gelidum on vacuum packaged frankfurters inoculated under controlled conditions.

Two strains of Carnobacterium piscicola NCIMB 702852 and UAL26 and Leuconostoc gelidum UAL187 were investigated.

For the preparation of inocula, bacteria from frozen culture were subcultured twice in APT broth (Difco) over 24 hours at room temperature. Cultures were centrifuged (10,000 rpm for 10 min, at 7° C.) and pelleted cells were resuspended with 0.85% sterile saline and washed three times by centrifugation. The final cell pellet was resuspended in 10 mL of sterile 0.85% saline to a final concentration of 1×10⁹ CFU per mL. Prior to dipping, a 10 mL aliquot of washed bacterial cells was added to 4 L of sterile 0.85% saline to provide an inoculum solution of 2.5×10⁶ CFU per mL. Groups of 5 frankfurters were dipped into the inoculum suspension for 1 minute, drain dried, and vacuum packaged (high barrier, low O₂ permeability bags). For control samples, frankfurters were dipped in 0.85% sterile saline without bacterial inoculum. Reference samples were frankfurters that had been dipped in a 0.85% saline solution and stored as described for treated samples.

Treated and control samples were placed into refrigerated 4° C. storage (monitored with a Delphi temperature recorder) for up to 12 weeks. Sampling of frankfurters for sensory evaluation was performed on day 0 and after 2, 4, 6, 7, 8, 10 and 12 weeks of storage.

Prior to sensory evaluation by a trained panel, frankfurters (4-5) were heated by placing in a saucepan containing 2 L of boiling tap water, which was immediately removed from the heat element and allowed to stand for 5 min (internal temperature approx. 83° C.). frankfurters were cut into 1.27 cm (0.5 inch) pieces and placed in coded foil-covered jars, and just prior to evaluation, heated for 15 min in a 200° F. (94° C.) oven (internal temperature approx. 66° C.).

Sensory evaluation was conducted by a group of 9 panelists trained over a three-month period, and was performed in sensory booths under dim red lighting using data collection software. Samples were evaluated for overall aroma intensity, meat flavor intensity, seasoned flavor, smoke intensity, sourness/acidity, off flavor and overall acceptability using a 15 cm unstructured line scale with 0=none (or very bland) and 15=extreme (or very strong). Between samples, palates were cleansed with crackers and a 1:1 dilution of 7-up.

All collected data were analyzed using the GLM of SAS version 6.12 (SAS Institute, 1996) and the Student Newman Keul's multiple range test was used to test for significant differences among treatments and storage times.

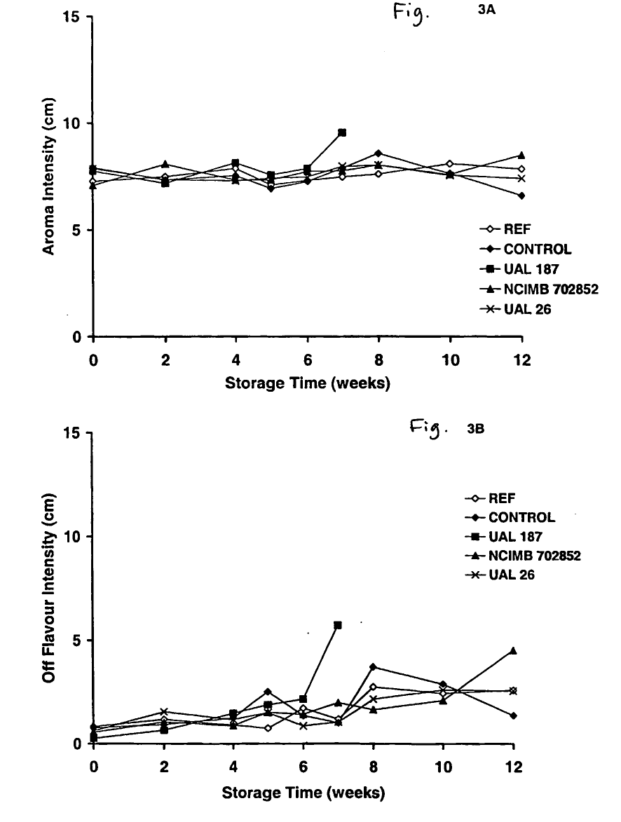

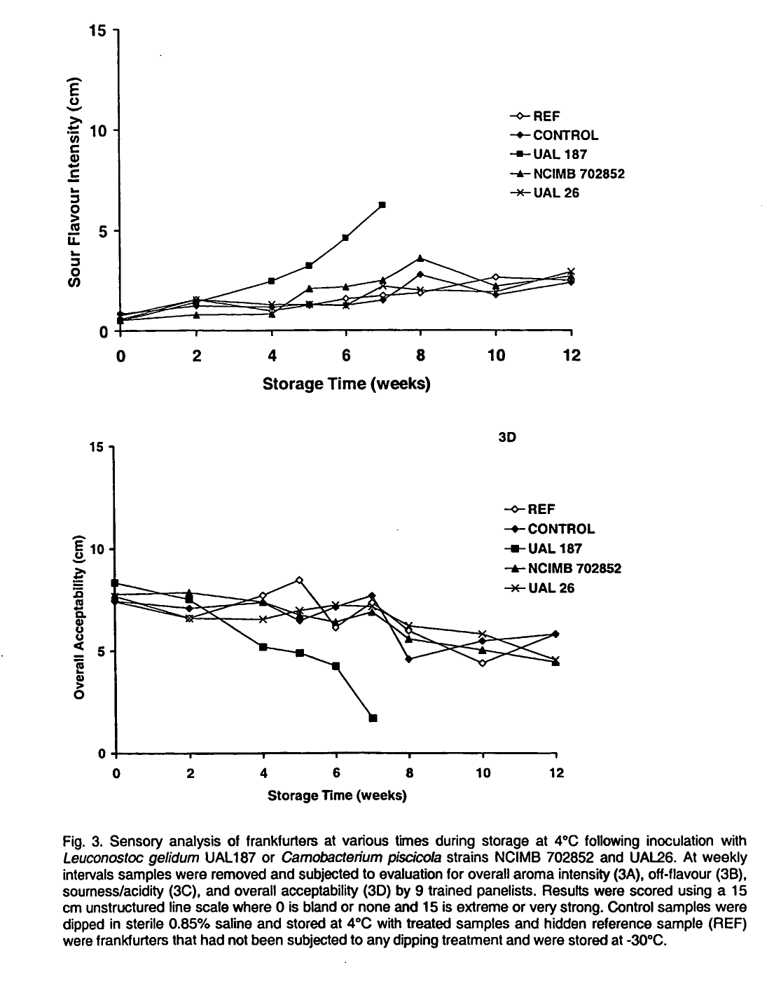

After 12 weeks of cold storage, frankfurters inoculated with Carnobacterium piscicola NCIMB 702852 and UAL26 were similar to control and reference samples for all characteristics. The results obtained are shown in FIG. 3. Samples inoculated with strain NCIMB 702852 had slightly higher off-flavor scores (4.5) at week 12 than samples inoculated with UAL26 (2.6). frankfurters inoculated with Leuconostoc gelidum UAL187 were unacceptable or spoiled by week 7 following inoculation and the samples were significantly different from the control samples in aroma, meaty, seasoning, smoky, sour and off-flavors.

Based on sensory evaluations using a trained nine-member panel over the 12-week storage period, there were no significant adverse effects on aroma, off-flavors, sour intensity, or overall acceptability resulting from inoculation of frankfurters with Carnobacterium piscicola.

Although a few preferred embodiments have been shown and described, it will be appreciated by those skilled in the art that various changes and modifications might be made without departing from the scope of the invention. The terms and expressions in the preceding specification have been used herein as terms of description and not of limitation, and there is no intention in the use of such terms and expressions of excluding equivalents of the features shown and described or portions thereof, it being recognized that the scope of the invention is defined and limited only be the claims that follow.

We claim:

1. A method of treating a food product against Listeria comprising contacting a food product with a composition comprising one or more bacteria cultures selected from the group consisting of NCIMB 702852, UAL26, CB1, CB2, CB3, and UAL 185; a fermentate from one or more of the above microorganisms; or combinations thereof.

2. The method of claim 1 wherein the food product is a processed meat.

3. A method of preserving foods comprising adding to the food or beverage an effective amount of a bacteriocin composition produced by the bacteria of claim 1.

4. A method of predicting spoilage of a food product comprising administering a known spoilage bacterium to a food product, and allowing the bacterium to provide a bactericidal or bacteristatic effect.

5. The method of claim 4 wherein administering a known spoilage bacterium further comprises administering a spoilage bacterium that is also effective against Listeria.

6. A composition for treating food comprising an effective amount of a microorganism selected from the group consisting of NCIMB 702852, UAL26, CB1, CB2, CB3, and UAL 185; a fermentate from one or more of the above microorganisms, or combinations thereof.

7. The composition of claim 6 wherein an effective amount is an amount that exceeds the level of natural contamination.

8. A food product treated with a composition comprising one or more bacteria cultures selected from the group consisting of NCIMB 702852, UAL26, CB1, CB2, CB3, and UAL 185; a fermentate from one or more of the above microorganisms, or combinations thereof.
